SEQUENCE LISTING (6 sequences)

SEQ ID NO: 1 - DNA, 39 bp, Artificial Sequence
  designed primer
catatggatccaagcttatgattctgtgcagttattggc

SEQ ID NO: 2 - DNA, 37 bp, Artificial Sequence
  designed primer
aggcctcgagaattcaatacgctgcatgataaaatgg

SEQ ID NO: 3 - DNA, 26 bp, Artificial Sequence
  designed primer
ggggaagcttatggcggcccccggcg

SEQ ID NO: 4 - DNA, 28 bp, Artificial Sequence
  designed primer
atgcaagctttagatcctcaaagagtgc

SEQ ID NO: 5 - DNA, 31 bp, Artificial Sequence
  designed primer
gggggaattcttgatccccattgctgtgggc

SEQ ID NO: 6 - DNA, 31 bp, Artificial Sequence
  designed primer
aaaactcgagctagatggtctgatagccggc

(19) United States
(12) Patent Application Publication
(10) Pub. No.: US 2005/0163762 A1
(43) Pub. Date: Jul. 28, 2005

(54) Targeting antigen-specific T cells for specific immunotherapy of autoimmune disease

(75) Inventor: Daniel B. Drachman, Stevenson, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD (US)

(21) Appl. No.: 10/871,515

(22) Filed: Jun. 21, 2004

Related U.S. Application Data
(63) Continuation of application No. 09/205,096, filed on Dec. 3, 1998, now abandoned.
(60) Provisional application No. 60/067,547, filed on Dec. 3, 1997.

(51) Int. Cl. A61K 48/00; A61K 39/395

(52) U.S. Cl. 424/93.21; 424/144.1

(57) ABSTRACT

Antigen-specific T cells are stimulated to proliferate by activation with properly presented antigen. Targeting a detrimental product to only those cells which are stimulated to proliferate in response to the antigen presentation permits the selective and highly efficient ablation of a key component of the disease symptomology of myasthenia gravis.

This application claims the benefit of provisional application No. 60/067,547 filed Dec. 03, 1997.

### BACKGROUND OF THE INVENTION

One of the major goals in immunotherapy is to devise methods that will effectively eliminate autoimmune responses in an antigen-specific manner, without otherwise affecting the immune system. Myasthenia gravis (MG), which is perhaps the most thoroughly characterized human autoimmune disease, is characterized clinically by weakness and fatigability of skeletal muscles. The pathogenesis of MG in humans and experimental MG in animals involves an antibody mediated autoimmune response directed against acetylcholine receptors (AChRs) at neuromuscular junctions. Although anti bodies are directly responsible for the loss of AChRs at neuromuscular junctions, therapeutic strategies directed at AChR-specific B cells are not practicable in ongoing disease. However, the AChR-antibody response is T cell dependent. Given the pivotal role of CD4+ cells in AChR antibody production, elimination or inactivation of these AChR-specific T cells might abrogate the autoantibody response, with resulting benefit. The key to a specific T cell based therapeutic strategy is to be able to target sufficient AChR-specific T cells. However, as in many other autoimmune diseases, the T cell response to the autoantigen in MG is highly heterogeneous. Not only do each individual's T cells respond to multiple epitopes, but there are significant differences in the patterns of epitopes to which different individuals' T cells respond. Even in rodents inbred to have highly restricted MHC Class II expression, there is significant heterogeneity in the AChR-specific T cell repertoires within a given strain, and even more marked differences in the repertoires in different strains.

Thus there is a need in the art for methods to effectively target AChR-specific T cells in patients with myasthenia gravis.

### SUMMARY OF THE INVENTION

It is an object of the invention to provide methods for activating auto-antigen specific T cells such as AChR-specific T cells in an auto-immune disease patient such as a myasthenia gravis patient.

It is another object of the invention to provide autologous antigen presenting cells which have been engineered to express, process and present an antigen to activate antigen-specific T cells of a myasthenia gravis patient.

It is still another object of the invention to provide a virus for transferring genes to antigen presenting cells so that they express, process, and present an antigen which will stimulate antigen-specific T cells in a myasthenia gravis patient.

These and other embodiments of the invention are provided by one or more of the embodiments described below. In one embodiment of the invention, a method is provided of activating AChR-specific T cells in a myasthenia gravis patient. Antigen presenting cells (APCs) are removed from a myasthenia gravis patient. The APCs are transduced or transfected with a gene which encodes all or a portion of an acetylcholine receptor (AChR) comprising at least the extracellular domain of the α subunit. The transduced or transfected APCs are reintroduced into the patient, whereby AChR-specific T cells are activated.

According to yet another aspect of the invention antigen presenting cells of an auto-immune disease patient are provided which are transfected or transduced to express a first segment of DNA encoding all or a portion of AChR comprising its α subunit's extracellular domain. The cells further comprises a second segment of DNA encoding a signal peptide 5′ to the first segment, as well as a third segment of DNA encoding a transmembrane and cytoplasmic tail which is 3′ to the first segment of DNA. The AChR protein expressed is appropriately processed by endosomes by virtue of the 5′ and 3′ signals.

Still another aspect of the invention is a virus which infects human APCs. The virus comprises a nucleic acid segment which encodes all or a portion comprising at least an extracellular domain of α-subunit of AchR. It will be readily understood by those of skill in the art that the method is also applicable to other auto-immune diseases, including systemic lupus erythematosus, multiple sclerosis, connective tissue disease, autoimmune pulmonary inflammation, Galliaume Barre Syndrome, autoimmune thyroiditis, insulin-dependent diabetes mellitus (IDDM), graft versus host disease, and rheumatoid arthritis.

The present invention thus provides the art with methods and biological reagents for treating and ameliorating the debilitating effects of myasthenia gravis.

### BRIEF DESCRIPTION OF THE DRAWINGS

FIG. 1. Chimeric gene construct of TAChR with LAMP-1 signal sequence (LAMP-1 sig) and LAMP-1 transmembrane and cytoplasmic domains (LAMP-1Tm/Cyt). a) Map of construct. The 3 components were amplified by PCR using the primers indicated in Methods, and high-fidelity pfu TAChR α subunit extracellular domain (TAChRα1-210) was amplified from plasmid PSS-2, and mouse LAMP-1 sig and tm/cy were amplified from pcDNA1. TAChRα1-210 was first subcloned into pcDNA3.1⁺ with Hind III at the 5′ end, and EcoR I at the 3′ end. The mouse LAMP-1 sig was then subcloned 5′ to the TAChRα1-210 with Hind III sites at both the 5′ and 3′ ends, and the correct orientation was confirmed by PCR, using relevant primers (see text). Finally, the mouse LAMP-1tm/cy domain was cloned 3′ to the TAChRα1-210 with EcoR I and Xho I sites at its 5′ and 3′ ends respectively. b) RT-PCR detection of chimeric gene mRNA expression. A20 cells were transfected with 50 μg of the linearized plasmid, and selected with G418 for 10 days. Total RNA was extracted and sscDNA was synthesized. The chimeric gene MRNA was detected by RT-PCR using the 5′ TAChRα subunit forward primer and 3′ reverse mouse LAMP-1 Tm/Cyt domain primer.

FIG. 2. Lymphoproliferation assay, demonstrating stimulation of the AChR-specific T cell line by antigen presenting transfected A20 clones. The mouse TAChR specific T-cell line was prepared as described using TAChR as antigen. Control wild type (WT) A20 B lymphoma cells and transfected A20 clones were irradiated with 15000 rad before being used for antigen presentation. Triplicate microwell cultures were pulsed with ³H-TdR on day 3, during the last 6-12 hrs of culture. Counts from wells containing only APCs were subtracted as background. FIG. 2a) AChR-specific T cell line proliferation (cpm), using different APCs (splenocytes; WT A20 cells; transfected A20 clones), without or with added TAChR. In this experiment, 2.5×10⁴ T cells, and 1×10³ APCs were added to each well. FIG. 2b) Dose-response graph, showing the ³H-TdR incorporation of AChR-specific T cells in response to stimulation by different numbers of transfected A20 cells.

FIG. 3 Inhibitory effect of recombinant CTLA4Ig on antigen presentation by transfected A20 cells. Lymphoproliferation assay was set up with 2×10⁴ transfected irradiated A20 cells or WT A20 cells as APCs. TAChR was added to some cultures as indicated in FIG. 3. Human CTLA4Ig (50 μg/ml) was added to cultures on day 0 as indicated. [³H]-TdR was added for the final 6 h of a 4 day culture. Inhibition of proliferation was produced as shown.

FIG. 4 Effect of soluble FasL or anti-Fas antibody on proliferation induced by exogenous or endogenous TAChR stimulation. AChR-specific T cells (5×10⁴/well) were incubated with irradiated APCs, either control WT A20 cells or A20 cells stably transfected as above (5×10⁴/well). AChR (2.5 μg/ml) was added to some of the wells as indicated. On day 3 of the cultures, FasL (50×10⁻⁹ g/ml) or antibody to Fas (50×10⁻⁹ g/ml) was added to triplicate wells as indicated. The cultures were pulsed with [³H]-TdR during the final 6 h of 4 day cultures.

### DETAILED DESCRIPTION OF THE INVENTION

The present invention is based on the discovery that autologous antigen presenting cells (APCs) can be transfected or transduced to express, process, and properly present an antigen to antigen-specific T cells. Moreover, upon proper presentation, the antigen-specific T cells are activated. Activation of the selected class of antigen-specific T cells permits this class to be distinguished from other T cells and for them to be selectively targeted for ablation.

Such antigen-specific T cell activation provides a model system in which drugs and treatments can be screened to identify those which are effective in arresting growth of or eliminating the antigen-specific T cells. Certain drugs and treatments have already been identified which have such effect, including administration of CTLA4Ig, a fusion protein which blocks the costimulatory factors B7-1 and -2, administration of Fas ligand, and administration of antibody to Fas. Moreover, such therapeutic agents can be supplied to the activated antigen-specific T cells in a variety of ways. For example, in addition to direct administration, cells which express and secrete the agents can be administered. In addition, genes which encode such agents can be supplied- to the antigen presenting cells, such that upon interaction of the antigen presenting cells and the antigen-specific T cells, the latter cells can be both activated and ablated. This technique can be used with any auto-immune disease in which the inciting auto-antigen is known and for which the coding sequence is available.

Antigen presenting cells can be withdrawn from an auto-immune disease patient, such as a myasthenia gravis patient according to techniques well known in the art. They can be found in the blood as well as in the bone marrow. In one embodiment of the invention B cells are purified from the blood and used as the preparation of antigen presenting cells.

All or a part of the coding sequence for acetylcholine receptor is transduced or transfected into the APCs. This can be accomplished by any technique known in the art, including using viral vectors or plasmid vectors. The extracellular portion of the α-subunit (comprising amino acids 1-120) is believed to comprise the epitopes to which most AChR-specific T cells respond. Thus this portion is believed to be sufficient as the presented antigen for the antigen presenting cells. Moreover, it is believed that proper processing signals are required for the antigen presenting cell to properly process the antigen and display it on its surface. Such processing is accomplished by the cellular endosomes. Proper signals on both the N-terminal and C-terminal portion of the protein are believed to be required. These can be supplied, inter alia, using the signal and transmembrane and cytoplasmic tail portions from LAMP-1 and LAMP-2 genes. Such signals are known in the art.

Transduced or transfected autologous antigen presenting cells are re-introduced to the patient using standard techniques for transfusing blood cells. The consequence of the introduction is that the acetylcholine receptor-specific T cells are activated.

Administration of an antigen-specific T cell-detrimental product can be by a variety of means. The product itself can be directly administered. Alternatively, cells which express and secrete the product can be administered. Another mode is to transduce or transfect the antigen presenting cells with a gene which expresses the detrimental product. Detrimental products which have been found to successfully inactivate or ablate activated antigen-specific T cells include CTLA4Ig, a fusion protein which binds to and blocks costimulatory B7 molecules on APC cells, Fas ligand, and antibodies to Fas itself. Antibodies which block costimulatory B7-1 and -2 molecules can also be used. It is known in the art that expression of Fas ligand by a cell can be suicidal. Thus if Fas ligand-expressing cells are to be used to administer Fas ligand, then a protective molecule such as truncated FADD should be used. These polypeptides are known in the art to protect expressing cells from the pro-apoptotic effects of Fas ligand.

Any viruses capable of introducing exogenous desired DNA to the antigen presenting cells can be used. For example, Vaccinia virus vectors can be used for transducing mature antigen presenting cells. Other non-viral techniques can be used, preferably those which are targeted to the proper cell targets, i.e., the antigen presenting. cells. It is preferred that the viruses be attenuated so that they do not replicate. One suitable means for attenuation is treatment with ultraviolet light, although other means as are known in the art may also be used.

In particular Vaccinia viruses with antigen have been made and used. These include Vaccinia with influenza hemagglutinin-LAMP 1, AchR(α1-210)-LAMP1, and Sig-AchR(α-210)-LAMP1. We have obtained cDNA for Fas ligand from pTK7.5b plasmid. We have made vaccinia viruses with HA-LAMP1 and Fas ligand; AchR(α1-210) and Fas ligand; and Sig-AChR(α1-210) and Fas ligand. Truncated FADD (NFD4) was obtained from pIV113. We have made vaccinia viruses with HA-LAMP 1 and Fas ligand and truncated FADD; AchR(α1-210) and Fas ligand and truncated FADD; and Sig-AChR(α1-210) and Fas ligand and truncated FADD. The Acetylcholine receptor is derived from Torpedo electroplax. LAMP1 is the transmembrane/cytoplasmic tail of LAMP1, which directs the antigen to the endosomal processing compartment. The signal sequence (Sig) is derived from LAMP1 and is necessary to direct the antigen across membranes. The truncated FADD is an amino terminal truncation which is a dominant negative that prevents Fas ligand/Fas mediated apoptosis.

Transduced or transfected antigen presenting cells, according to the invention are in vitro preparations into which DNA has been inserted ex vivo. Preferably the cells are purified to remove T cells or other cells which are not antigen presenting cells, or other cells which have not been transduced.

The following is provided as exemplification. The scope of the invention is not defined by the scope of the examples only. All references cited are hereby incorporated by reference herein.

### EXAMPLES

A line of APCs derived from BALB/c mice has been engineered to process and present the most important AChR epitopes. The A20 B lymphoma cell line has been transfected with a gene construct coding for the extracellular portion of the a subunit of AChR together with signals that direct it to the endosomal processing compartments. These transfected APCs strongly target and stimulate AChR-specific T cells from BALB/c mice. Further, this endogenous APC-driven stimulation has combined with agents that interfere with T cell function. When stimulated in the presence of CTLA4Ig, the T cells are relatively inactivated; when stimulated in the presence of Fas ligand or antibody to Fas they undergo apoptosis and death. Thus genetic engineering of APCs permits targeting the spectrum of T cells specific for the autoantigen AChR, and permits inactivation or elimination of these T cells.

### MATERIALS AND METHODS

Animals and Reagents

Female inbred BALB/c mice, 8-12 weeks of age, from Charles River Labs (Wilmington, Mass.) were used in this study. Acetylcholine receptor (TAChR) was purified from the electric organs of Torpedo californica (Pacific Biomarine, Venice Calif.) by affinity chromatography, using α cobra toxin linked to Sepharose 4B beads, as previously described. The CTLA4Ig fusion protein was generously provided by Bristol-Meyers Squibb Pharmaceutical Research Institute (Princeton, N.J.). Plasmids with cDNA for the α subunit of TAChR were a kind gift from Dr. Toni Claudio. The cDNA for mouse LAMP-1 was a gift from Dr. T. August. The B cell lymphoma line A20, derived from BALB/c mice, was purchased from ATCC.

Immunization of Mice

To produce lymph node cells primed to TAChR (pLNC), BALB/c mice were immunized intradermally with TAChR (50 μg emulsified in complete Freund's adjuvant), in multiple sites over the low back. Two weeks later, the draining inguinal lymph nodes were removed, and processed into single-cell suspensions as described previously.

Preparation of Short-Term T Cell Lines

In order to confirm the antigen responsiveness of the pLNC from which T cell lines would be produced, the responses of pLNC to AChR in vitro were first tested as follows: pLNC were cultured in flat-bottomed 96 well microtiter plates (Costar, Cambridge, Mass.) at a density of 5×10⁵ cells/well. Complete culture medium consisted of: RPMI 1640 containing 2 mM 1-glutamine, supplemented with 5% fetal bovine serum (FBS, Hyclone Labs, Logan, Utah), 5×10⁻⁵ M 2-mercaptoethanol, 1 mM sodium pyruvate, 0.1 mM nonessential amino acids, and 1:100 of 100× penicillin-streptomycin-fungizone (BRL, Gaithersburg, Md.). Cells were cultured for 4 days in the presence of an optimum stimulating concentration of TAChR (2.5 μg/ml), and unstimulated cultures were used for subtraction of background responses.

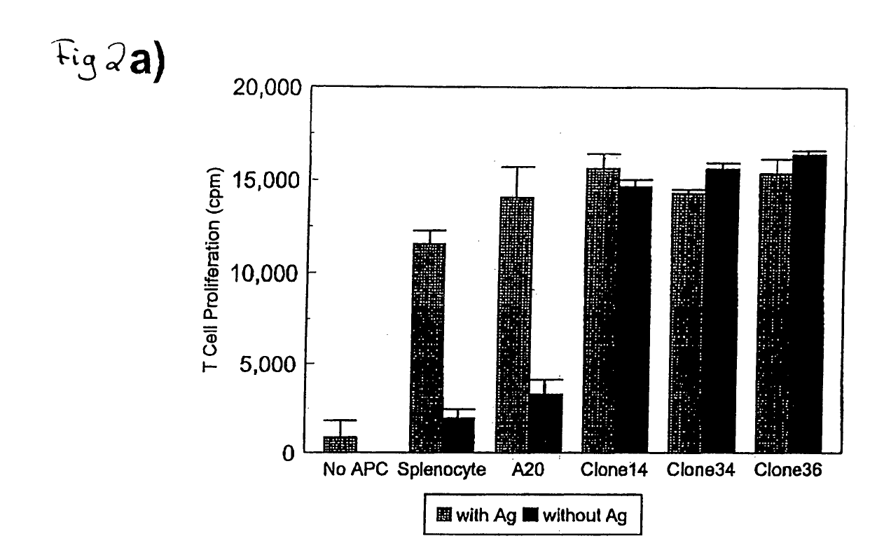

Short-term T-cell lines specific for TAChR were produced as described previously. Briefly, pLNC (5×10⁶/ml) were stimulated with TAChR (2.5 ug/ml) in bulk cultures (100×10⁶ pLNC/100 mm plastic tissue culture dish in 20 ml)for 4 days. The activated T cells (2×10⁵ cells/ml) were expanded in complete medium containing recombinant human IL2 (rhIL2, 100 IU/ml) and 10% FBS for 5-7 days. The resulting population, which consisted primarily of T cells, responded to AChR stimulation only when antigen presenting cells (APCs) were added, but showed negligible [³H]-TdR incorporation in the absence of added APCs (mean cpm of T cell lines without APCs in 5 different experiments=587±200; significantly different from AChR-stimulated T cell lines cultured with APCs [p<0.005] see FIG. 2).

Cloning and PCR.

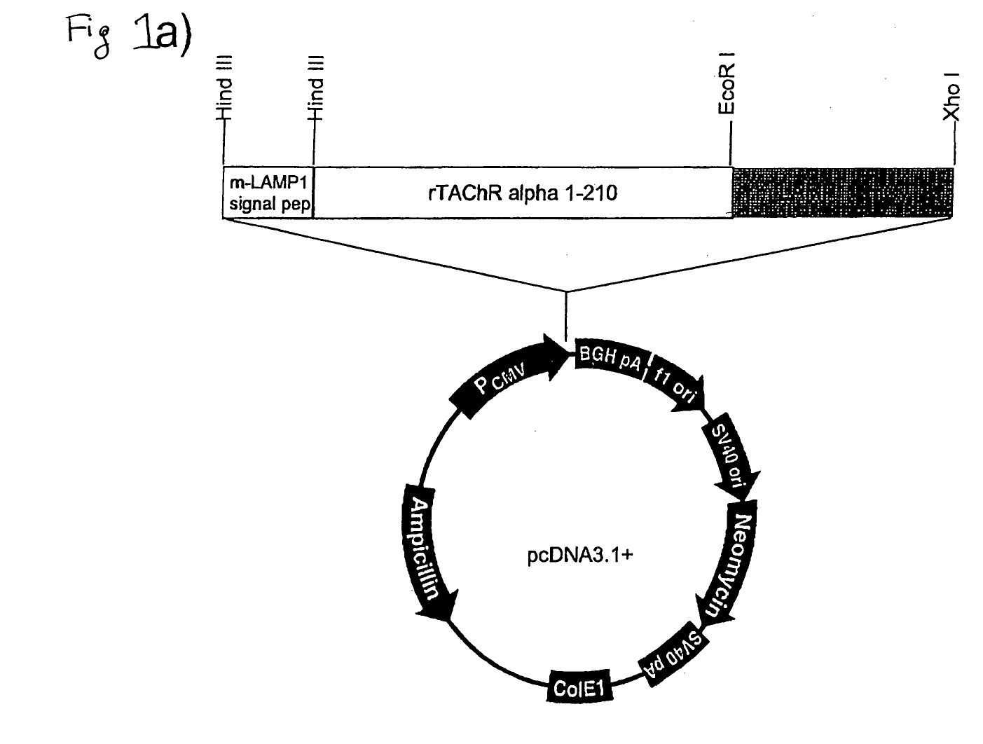

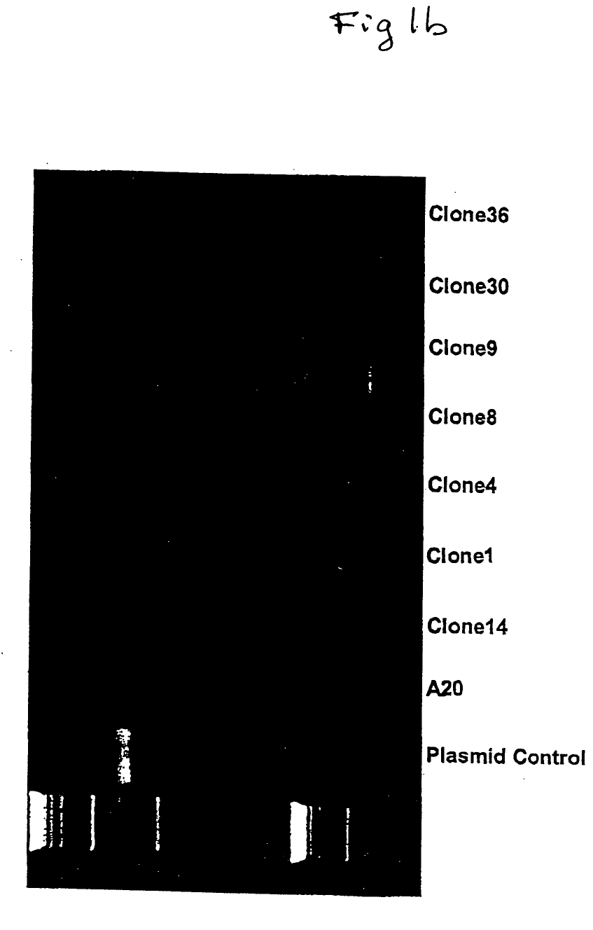

The construct used for transfection consisted of cDNA for the extracellular sequence of the TAChRα subunit (amino acids 1-210), with the mouse LAMP-1 signal sequence (LAMP-1sig) at its 5′ end, and the mouse Lamp-1 transmembrane and cytoplasmic tail (LAMP-1 Tm/Cyt) at its 3′ end, cloned into a mammalian expression vector, pcDNA3.1+ plasmid (Invitrogen, Carlsbad, Calif.) carrying neomycin resistance as a selectable marker (FIG. 1a). To evaluate the role of the LAMP-1 signal peptide in AChR antigen processing and presentation, a vector without the cDNA for Lamp-1 sig, but with cDNA that coded for TAChR+LAMP-1Tm/Cyt was also prepared. Sequential cloning methods were used, with TAChRα1-210 first cloned with both adhesive ends (Hind III at the 5′ end, and EcoR I at the 3′ end); followed by mouse LAMP-1 sig (both ends with Hind III), and finally mouse LAMP-1 Tm/Cyt domains (EcoR I at the 5′ end, and Xho I at the 3′ end) cloned into this mammalian expression vector. Each of the insert fragments was first ampified by PCR, using related primers as follows:

TAChR alpha1-210aa
5′ CAT ATG GAT CCA AGC TTA TGA TTC TGT GCA
GTT ATT GGC
3′ AGG CCT CGA GAA TTC AAT ACG CTG CAT GAT
AAA ATG G
mouse LAMP-1 sig
5′ GGG GAA GCT TAT GGC GGC CCC CGG CG
3′ ATG CAA GCT TTA GAT CCT CAA AGA GTG C
mouse LAMP-1 Tm/Cyt
5′ GGG GGA ATT CTT GAT CCC CAT TGC TGT GGG C
3′ AAA ACT CGA GCT AGA TGG TCT GAT AGC CGG C

PCR ampification of these fragments was carried out using the high fidelity pfu polymerase, under the following condition: 94° C.-3 min for one cycle; then 94° C., 1 min/60° C., 2 min/72° C. for 30 cycles; and finally, 72° C. for 10 min. Fragments were digested with restriction enzymes, and the DNA was purified using a QIAquick gel extraction kit (Qiagen, Chatsworth, Calif.). DNA ligation reactions were carried out as described. The inserts and the oriention of the mouse LAMP-1 sig were confirmed by PCR with related primers. The sequence of the entire construct was confirmed in the Johns Hopkins Genetic Core facility by the dideoxy chain termination method.

Transfection and Selection of A20 Cells.

A20 B lymphoma cells were obtained from ATCC, and grown in culture. The critical parameters for transfection by electroporation were determined empirically to be 350 volts and 500 uF capacitance. A20 cells in log phase were harvested, washed ×3 with cold serum-free medium, and kept on ice for 15 min prior to electroporation. For each electroporation 8×10⁶ A20 cells were mixed with 50-100 μg of linearized plasmid in 0.8 ml of serum-free RPMI 1640 at 4° C. They were kept on ice for an additional 15 min., and incubated at 37° C. in 20 ml of recovery medium (20% FBS in complete RMPI 1640 medium). Two days later, transfected cells were selected and maintained with 1 mg/ml of G418 in RMPI 1640 with 10% FBS. Transcription of the chimeric mRNA in transfected A20 cells was checked by RT-PCR as described elsewhere.

Antigen Presentation: Lymphoproliferation Assay.

Antigen presenting cells were first irradiated with a ⁵⁰Co gamma irradiator before use. Splenocytes were treated with 1200 rad at a cell concentration of 5×10⁶/ml in RPMI 1640 with 10% FBS. A20 cells (either wild type [WT] or transfected) were treated with 15,000 rad ( ).

Briefly, 2.5×10⁴ or 5×10⁴ cells of the short-term T-cell lines were added to each well. The numbers of APCs added were 2-5×10⁵/well for splenocytes; or 200 to 25,000/well for WT A20 cells or transfected A20 clones. Triplicate cultures for each treatment were incubated for 3 days in flat-bottom 96-well microculture plates at 37° C. The optimal concentration of antigen (2.5 μgTAChR/ml) was used for exogenous antigen stimulation. To assess lymphoproliferation, the cultures were pulsed with [³H]-TdR (1 uCi/well, 50ul) during the final 6-12 hr of a 3-4 day culture period. All cultures were harvested on filters (PHD Cell Harvester, Cambridge Technology, Cambridge, Mass.) for scintillation counting. Radioactivity incorporated by APCs alone, unstimulated T cell line cultures, and T cell lines cultures with added AChR, was counted, and results are expressed as CPM.

Assay for Killing of Antigen-stimulated T Cells by Fas Ligand or Antibody to Fas.

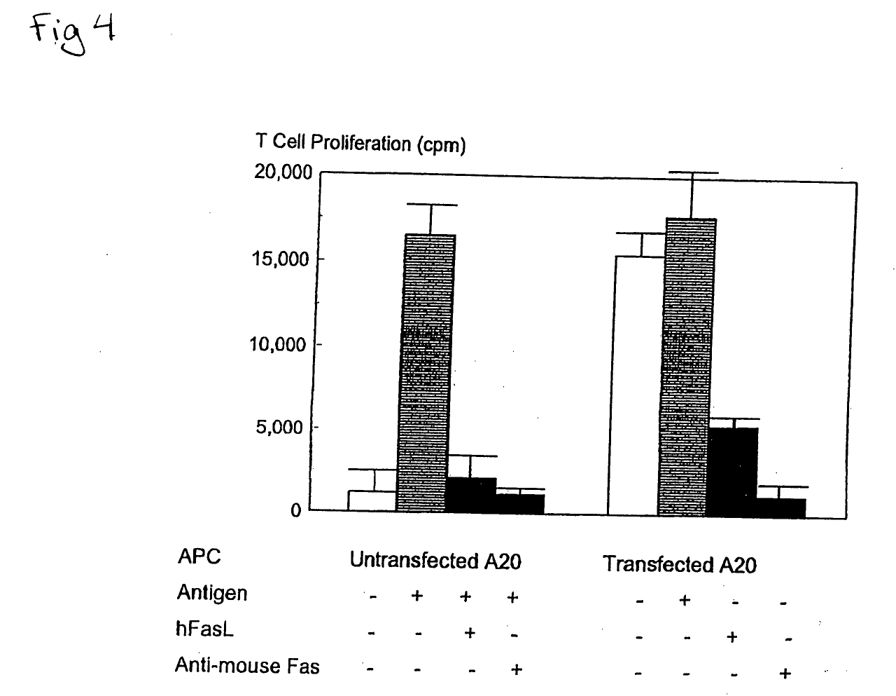

AChR-specific T cells (5×10⁴/well) were incubated with irradiated APCs, either control WT A20 cells or A20 cells stably transfected as above (5×10⁴/well). AChR (2.5 μg/ml) was added to some of the wells as indicated in FIG. 4. On day 3 of the cultures, FasL (50×10⁻⁹ g/ml, Calbiochem, Cambridge, Mass.), or antibody to Fas (50×10⁻⁹ g/ml, Pharmingen, Inc., San Diego, Calif.) was added to triplicate wells as indicated (FIG. 4). The cultures were pulsed with [³H]-TdR during the final 6 h of 4 day cultures. All studies were carried out in triplicate wells. A typical result is shown in FIG. 4.

### RESULTS

Transcription and Expression of Gene Construct

RT-PCR was performed on total RNA from transfected A20 clones to determine whether stably transfected A20 cells had transcribed the chimeric gene. Using the forward primer for the 5′ end of TAChR and the reverse primer for the 3′ end of the mouse LAMP-1 Tm/Cyt domain, the presence of the appropriate mRNA (FIG. 1b) was demonstrated.

Functional testing was performed to evaluate the antigen presenting ability of transfected A20 clones. The transfected clones stimulated AChR-specific T cell lines vigorously, without the addition of exogenous AChR (FIG. 2). Addition of optimal concentrations of AChR to cultures with these transfected clones did not further enhance stimulation, indicating that the stimulating effect of the transfected clones was maximal. Dose response curves indicated that 200 to 1,000 transfected A20 cells maximally stimulated 2.5×10³ AChR-specific T cells in culture (FIG. 2b). By contrast, when WT A20 cells or splenocytes were used as APCs, stimulation of the AChR-specific T cell line occurred only when exogenous TAChR was added to the cultures.

The ability of A20 clones transfected with cDNA for TAChR plus the LAMP-1 Tm/Cyt domain—but not the signal peptide sequence—to stimulate AChR-specific T cell hybridomas or T cell lines was tested. Only background [³H]-TdR incorporation was induced by these transfectants, unless exogenous TAChR was added to the cultures (data not shown). These results indicate that APCs can be modified by genetic engineering to process and present the “autoantigen” AChR, provided that the gene for the antigen as well as the genes for the required peptide sequences to direct it to the processing lysosomal compartments are in place.

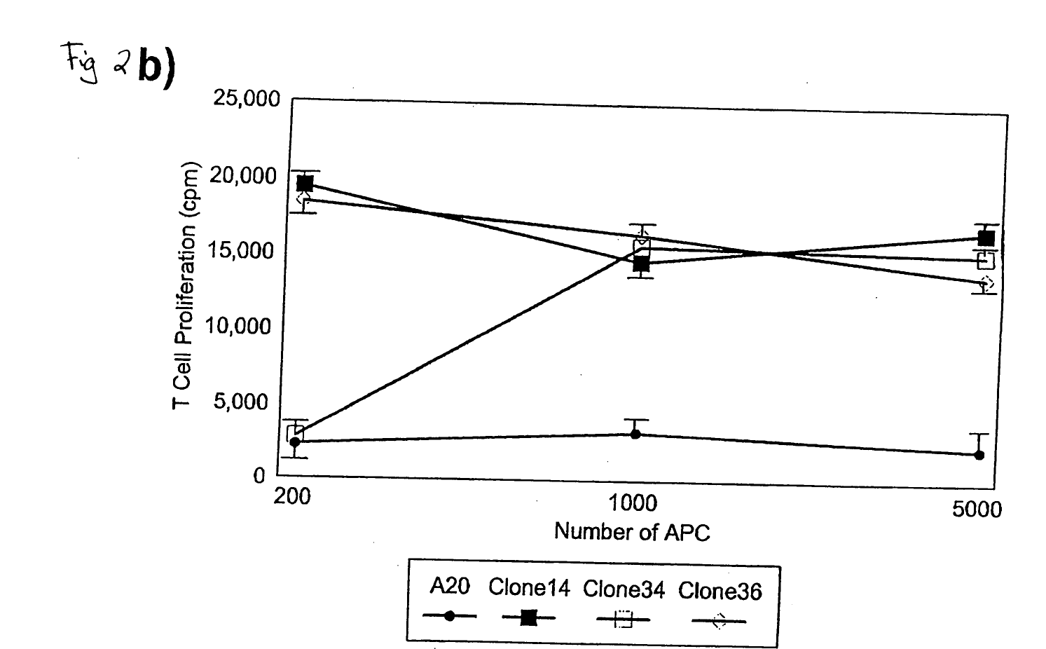

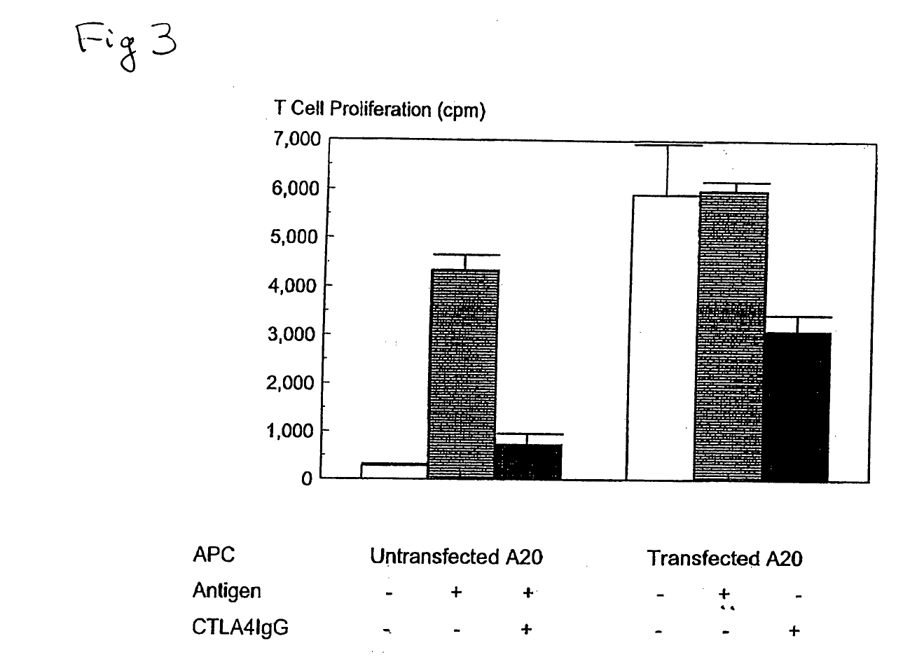

To examine the ability of these APCs to be used to inactivate—rather than stimulate—the AChR-specific cells, endogenous stimulation of the target T cells by the transfected APCs was combined with simultaneous treatment with CTLA4Ig (to block costimulation). Endogenous stimulation by transfected A20 cells in the presence of 50 μg/ml of CTLA4Ig resulted in 70% inhibition of stimulation (FIG. 3), which is consistent with results of previous studies of in vitro effects of CTLA4Ig on AChR-specific T cell stimulation.

The ability of Fas ligand (FasL) and antibody to Fas to induce killing of AChR-specific T cells that were stimulated by transfected APCs (FIG. 4) was tested. In order to allow the transfected APCs to complete their stimulation of the T cells, and to avoid possible interference with the APCs, FasL or anti-Fas antibody were first added 72 hrs after initiation of the cultures. Antibody to Fas resulted in virtually 100% inhibition of T cell proliferation, and treatment with soluble hFasL produced 70 to >90% inhibition. See FIG. 4. In these experiments, T cells were stimulated equally strongly by transfected A20 cells presenting endogenous antigen and by control WT A20 cells with added exogenous AChR. Stimulation by transfected A20 cells was not significantly enhanced by the addition of exogenous AChR. The key point is that both FasL and anti-Fas antibody dramatically inhibited T cells that were stimulated either by endogenously or by exogenously presented AChR.

The development of a strategy for specific immunotherapy of autoimmune disease must meet several challenges. Ideally, it should interfere selectively with the pathogenic autoimmune response, without otherwise compromising the remainder of the immune system. Of necessity, it must cope with ongoing immune responses, which are notoriously difficult to suppress, particularly in an antigen-specific manner. The treatment should inactivate or eliminate the lymphocytes that are responsible for the disease process, and its effect should be long-lasting or permanent. Thus far, the goal of specific immunotherapy has remained elusive. A major problem in designing specific immunotherapy is the heterogeneity of the autoreactive lymphocytes, which recognize multiple epitopes of the autoantigen. Thus, in MG, each individual's T cells respond to multiple epitopes of the autoantigen AChR, and there are marked differences in the patterns of epitopes to which different individuals' T cells respond. Even in rodents inbred to have highly restricted MHC Class II expression, there is significant heterogeneity in the AChR-specific T cell repertoires within a given strain, and even more marked differences in the repertoires in different strains.

The present invention is based on the development of a method of targeting the extensive spectrum of T cells that recognize the autoantigen AChR in MG. The principle underlying the approach is to use the individual patient's own APCs to target the AChR-specific T cells, since the APCs naturally interact with the entire spectrum of antigen-specific T cells for that individual. Modifications are made to induce inactivation or elimination of the targeted T cells, instead of stimulation.

APCs can be engineered to process and present a model autoantigen, and will effectively target the spectrum of T cells related to autoimmune disease. In these experiments the moiety of TAChR to which the majority of T cells respond—i.e.—the extracellular 1-210 amino acid sequence of the α subunit—as the antigen was used. Endogenous presentation by transduced APCs was equivalent to presentation of exogenous AChR, in terms of eliciting T cell proliferative responses.

Effective presentation requires not only cDNA for the autoantigen, but also the appropriate signals to direct it to the endosomal processing compartment. Both LAMP-1 sig and LAMP-1 Tm/Cyt are required; without either component the antigen is not be efficiently processed and presented. TAChR+LAMP-1 Tm/Cyt (without sig) were tested, and found virtually no stimulation. When LAMP-1 sig was cloned in to the construct, vigorous stimulation occurred, as above. In a previous study, presentation of influenza hemagglutinin (HA) required the LAMP-1 Tm/Cyt domain to direct it to the endosomal processing compartment. Since the HA molecule carries its own signal sequence, this was apparently sufficient. Thus, it is clear that both components are necessary to direct the endogenously produced protein antigen to the processing endosomal compartment.

The effects of endogenous autoantigen presentation can be modified by immunomodulatory treatments in the same manner as though the antigen were derived from an exogenous source. Two different methods were tested for interfering with stimulated T cells: blockade of the costimulatory signals provided by B71 and B72, using recombinant CTLA4Ig; and activation of the Fas system, by means of FasL or antibody to Fas.

CTLA4Ig is a soluble fusion protein consisting of the extracellular domain of the CTLA4 receptor (for B7-1 and B7-2), and the Fc portion of human IgG1 (added for solubility []) CTLA4Ig binds with extremely high affinity to the B7 family of ligands on APCs, and blocks them, thereby preventing the delivery of these costimulatory signals to T cells. CTLA4Ig has been shown to prevent and treat certain autoimmune diseases, including EAMG. Because of the potential usefulness of this approach in the treatment of MG, and because of the availability of the gene for CTLA4Ig, it was tested in our system. CTLA4Ig inhibited AChR-specific T cell proliferation by ˜70%, which is consistent with previous results in vitro. In the present experiments, the inhibitory effect of CTLA4Ig was somewhat greater when using wild type A20 cells with exogenously provided AChR. This is attributable to weaker stimulation from the exogenously provided TAChR. In previous studies the inhibitory effect of CTLA4Ig on proliferation was greater with weaker stimulation (lower antigen concentrations or fewer APCs).

It has been shown that activated T cells express high levels of the Fas receptor molecules on their surface membranes, but resting T cells do not. Interaction with the cross-linking molecules FasL or antibody to Fas, induces apoptosis of the Fas-bearing cells. FasL and antibody to Fas were supplied exogenously. Both reagents were highly effective in inhibiting AChR-specific T cell proliferation, presumably by inducing Fas-mediated apoptosis. In order to test the effects of these agents on the T cells alone, without damaging APC function, FasL or Fas Ab were added on the 3rd day of the cultures, after maximal stimulation had already occurred. [³H] TdR incorporation was decreased by >70-90% after FasL treatment, and by virtually 100% after treatment with anti-Fas antibody.

These results indicate that the methods of treating myasthenia gravis can be utilized for antigen-specific immunotherapy. The goal is to focus the immunotherapeutic effect at the level of the pivotal autoreactive T lymphocytes. In the case of MG (or EAMG), where the antigen is known to be AChR, the pivotal cells are AChR-specific T cells. Since AChR antibody production is T cell dependent, inactivation or elimination of AChR-specific T cells interferes with AChR antibody production without otherwise altering the function of the immune system.

The genes of interest have been transferred by transducion to demonstrate that the autoantigen can be effectively presented so as to target the appropriate antigen-specific T cells. In order to test the principle of using targeting APCs together with immunomodifying reagents, CTLA4Ig, FasL, and anti-Fas antibody, from exogenous sources were supplied. However, a more efficient method of focusing both antigen presentation and the modifying reagent(s) is to use a viral vector which can transfer all the necessary genes for both functions.

1. A method of activating auto-antigen-specific T cells in an auto-immune disease patient, comprising the steps of:
removing antigen presenting cells (APCs) from an auto-immune disease patient;
transferring into the APCs a gene which encodes all or a portion of an auto-antigen to which the patient's antigen-specific T cells respond; and
reintroducing the APCs into the patient, whereby auto-antigen-specific T cells are activated.

2. The method of claim 1 wherein the gene which encodes all or a portion of auto-antigen further comprises a signal sequence and a transmembrane and cytoplasmic tail sufficient for endosomal processing.

3. The method of claim 1 further comprising the step of:
administering a product which is detrimental to activated T cell proliferation or survival to the patient.

4. The method of claim 3 wherein the product is CTLA4Ig, a fusion protein which binds to and blocks costimulatory B7 molecules on APC cells.

5. The method of claim 3 wherein the product is a cell which expresses and secretes CTLA4Ig.

6. The method of claim 3 wherein the product is Fas ligand.

7. The method of claim 6 wherein the Fas ligand is administered by administration of APC cells which express Fas ligand.

8. The method of claim 6 wherein the APC cells which express Fas ligand also express A truncated form of FADD which protects cells producing the truncated form of FADD from the apoptotic effects of Fas ligand.

9. The method of claim 3 wherein the product is an antibody specific for Fas.

10. The method of claim 9 wherein the antibody specific for Fas is a monoclonal antibody.

11. The method of claim 9 wherein the antibody specific for Fas is a single chain Fv (ScFv) antibody.

12. The method of claim 11 wherein the antibody is administered by administration of APC cells which express the single chain Fv antibody.

13. The method of claim 8 wherein the APC cells which express Fas ligand and a truncated form of FADD are the same cells which express auto-antigen.

14. The method of claim 5 wherein the APC cells which express CTLA4Ig are the same cells which express auto-antigen.

15. The method of claim 7 wherein the APC cells which express Fas ligand are the same cells which express auto-antigen.

16. The method of claim 1 wherein the gene is transferred with a virus.

17. The method of claim 16 wherein the virus is attenuated.

18. The method of claim 16 wherein the virus is a vaccinia virus.

19. The method of claim 16 wherein the virus is a Moloney Leukemia Virus.

20. The method of claim 16 wherein said virus further encodes a product which is detrimental to activated T cell proliferation or survival.

21. The method of claim 20 wherein the product is Fas ligand.

22. The method of claim 20 wherein the product is a single chain Fv which blocks costimulatory B7 molecules.

23. The method of claim 21 wherein the virus further encodes a truncated form of FADD sufficient to protect a cell expressing it from the apoptotic effects of Fas ligand.

24. Antigen presenting cells of an auto-immune disease patient which are transduced or transfected to express a first segment of DNA encoding all or a portion of auto-antigen to which the patient's antigen-specific T cells respond, wherein the cells comprise a second segment of DNA encoding a signal peptide 5′ to said first segment and a third segment of DNA encoding a transmembrane and cytoplasmic tail 3′ to said first segment, whereby the encoded all or a portion of auto-antigen is processed by endosomes.

25. The antigen presenting cells of claim 24 which are transduced or transfected to express a protein which is detrimental to activated T cell survival or proliferation.

26. The antigen presenting cells of claim 25 wherein the detrimental protein is Fas ligand.

27. The antigen presenting cells of claim 26 which have been transduced to express a truncated form of FADD sufficient to protect a cell expressing it from the anti-apoptotic effect of Fas ligand.

28. The antigen presenting cells of claim 25 wherein the detrimental protein is a ScFv which blocks costimulatory B7 molecules.

29. A virus which infects human APCs and which comprises a first segment which encodes all or a portion comprising an epitope of an auto-antigen to which auto-immune disease patient's antigen-specific T cells respond.

30. The virus of claim 29 which is a vaccinia virus.

31. The virus of claim 29 which is a Moloney leukemia virus.

32. The virus of claim 29 further comprising a second segment which encodes a signal peptide 5′ to said first segment and a third segment encoding a transmembrane and cytoplasmic tail 3′ to said first segment, whereby the encoded all or a portion of auto-antigen is processed by endosomes.

33. The virus of claim 32 further comprising a fourth segment which encodes a product detrimental to proliferation or survival of activated T cells.

34. The virus of claim 33 wherein the product is Fas ligand.

35. The virus of claim 33 wherein the product is a ScFv which blocks costimulatory B7 molecules.

36. The virus of claim 34 further comprising a fifth segment which encodes a portion of FADD which is sufficient to protect a cell expressing Fas ligand from apoptosis.

37. The virus of claim 29 which is attenuated.

38. The method of claim 1 wherein the auto-antigen is extracellular domain of α-subunit of acetylcholine receptor and the auto-immune disease is myasthenia gravis.

39. The antigen presenting cells of claim 24 wherein the auto-antigen is extracellular domain of α-subunit of acetylcholine receptor and the auto-immune disease is myasthenia gravis.

40. The virus of claim 29 wherein the auto-antigen is extracellular domain of α-subunit of acetylcholine receptor and the auto-immune disease is myasthenia gravis.
